(19) United States
(12) Patent Application Publication
(10) Pub. No.: US 2025/0087366 A1
(43) Pub. Date: Mar. 13, 2025

(54) Extracting Biomarkers For Stress Monitoring Using Mobile Devices

(71) Applicant: Samsung Electronics Co., Ltd., Suwon-si (KR)

(72) Inventors: Md Mahbubur Rahman, Santa Clara, CA (US); David Lin, El Cerrito, CA (US); Li Zhu, Saratoga, CA (US); Viswam Nathan, Clovis, CA (US); Larry Zhang, San Jose, CA (US); Ramesh Sah, Mountain View, CA (US); Mehrab Bin Morshed, Gilroy, CA (US); Jungmok Bae, Menlo Park, CA (US); Jilong Kuang, San Jose, CA (US)

(21) Appl. No.: 18/824,767

(22) Filed: Sep. 4, 2024

Related U.S. Application Data
(60) Provisional application No. 63/537,431, filed on Sep. 8, 2023.
(60) Provisional application No. 63/537,681, filed on Sep. 11, 2023.

(51) Int. Cl. G16H 50/30; A61B 5/0205; A61B 5/024; A61B 5/11

(52) U.S. Cl. CPC G16H 50/30; A61B 5/0205; A61B 5/02405; A61B 5/02416; A61B 5/1102

(57) ABSTRACT

In one embodiment, a method includes accessing a window of motion data collected by a motion sensor of a device worn by a user and selecting motion data in the window about or more particular axes of the motion sensor. The method further includes determining a ballistocardiogram (BCG) signal in the selected motion data and determining whether the BCG signal in the selected motion data satisfies a signal-quality metric. If the BCG signal in the selected motion data satisfies the signal-quality metric, then the method includes (1) determining one or more heart-beat metrics of the user from the selected motion data; and (2) estimating, based on the one or more determined heart-beat metrics, a stress condition of the user at a time coincident with the window of motion data.

### PRIORITY CLAIM

This application claims the benefit under 35 U.S.C. § 119 of U.S. Provisional Patent Application Nos. 63/537,431, filed Sep. 8, 2023, and 63/537,681, filed Sep. 11, 2023, each of which is incorporated by reference herein.

### TECHNICAL FIELD

This application generally relates to extracting biomarkers for stress monitoring using a mobile device.

### BACKGROUND

Many different circumstances in a person's daily life can cause stress. Stress results in a set of physiological responses that may be detrimental to mental and physical well-being or may be useful to increase focus and productivity. For example, temporary stress over an important event or due to an emergency situation can increase awareness, responsiveness, alertness, etc., which may be beneficial to performance or to resolving the situation. Stress can also have a negative impact on physical and mental well-being, particularly if stress is present for relatively longer periods. For example, repeated or continuous stress over a person's job, interpersonal relationships, or other life circumstances can accumulate and is linked to cardiovascular diseases and essential hypertension.

Stress causes what is known as a “flight or fight” response. Physiological arousal due to stress affects heart-beat frequency and blood volume pulse, among several other bodily responses. The sympathetic nervous system's response to stress increases heart rate, while the parasympathetic nervous system calms the body down to maintain sympathovagal balance. The physiological responses due to stress and the negative long-term effects they induce can build up silently with little to no detectable symptoms until the user's health is negatively affected, and daily life stressors are often not easily identifiable.

### BRIEF DESCRIPTION OF THE DRAWINGS

FIG. 1 illustrates an example method for determining a stress condition of a user using passive, continuous sensing.

FIG. 2 illustrates an example approach for implementing the method of FIG. 1.

FIG. 3 illustrates examples of signal processing to extract BCG data from a motion signal as well as examples of signal-quality techniques to determine whether the BCG data is sufficiently accurate.

FIG. 4 illustrates an example of probabilistic estimator fusion of a BCG signal.

FIG. 5 illustrates an example in which data from multiple axes of an earbud is fused to estimate a BCG signal.

FIG. 6 illustrates an example IBI-estimate enhancement that uses heart-rate information and a prior IBI determination.

FIG. 7 illustrates examples of good-quality BCG signals and examples of poor-quality BCG signals.

FIG. 8 illustrates an example heatmap made by plotting the BCG beat relative to the corresponding ECG beat.

FIG. 9 illustrates an example process for estimating IBI and corresponding heart-beat metrics, such as HR and HRV, based on a PPG signal.

FIG. 10 illustrates an example PPG signal with multiple feet.

FIG. 11 illustrates an example of a high-quality PPG signal and corresponding derivatives.

FIG. 12 illustrates an example computing system.

### DESCRIPTION OF EXAMPLE EMBODIMENTS

Continuous physiological stress measurement combined with stress categorization (e.g., determining whether the stress is detrimental or favorable) can be useful for developing better stress management technologies.

Stress is typically detected by assessing cortisol in bodily fluid (e.g., saliva) or through a person's self-reports. The variety of physiological responses to stress makes it possible to capture and track a range of physiological signals such as electrocardiogram (ECG), respiration, and skin conductance responses, but because stress affects different organs differently, multiple sensing modalities in multiple devices placed in different part of the body are needed to capture the multi-modal stress response. This type of setup is both expensive and inconvenient for daily use. Furthermore, existing stress detection models typically cannot distinguish between positive stress responses and negative stress responses, since the sympathetic nervous system's response (e.g., heart rate, GSR) can be similar in both cases.

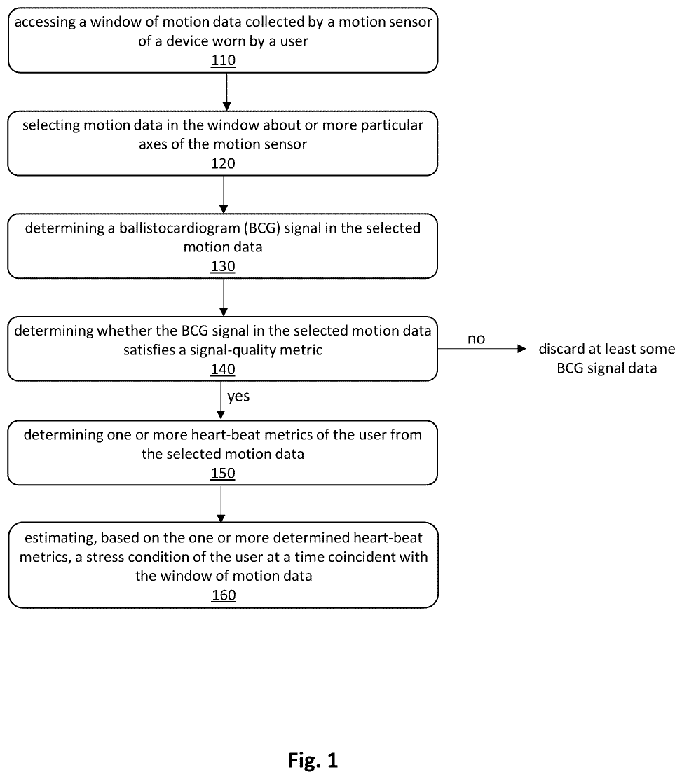

In contrast, the techniques of this disclosure estimate stress biomarkers using one or more built-in sensors of a consumer mobile device, such as a pair of earbuds. As described below, the estimated biomarkers include biomarkers that indicate a stress event (i.e., whether a stress arousal has occurred) and biomarkers that can be used to reliably classify the stress arousal, e.g., as negative or positive stress. As described below, embodiments of this disclosure can reliably estimate the stress biomarkers using low-power, passive continuous sensing. FIG. 1 illustrates an example method for determining a stress condition of a user using passive, continuous sensing.

Step 110 of the example method of FIG. 1 includes accessing a window of motion data collected by a motion sensor of a device worn by a user. In particular embodiments, the device may be a head-worn device, and this disclosure throughout uses examples in which the wearable device is a pair of earbuds or other headphones. Other embodiments may use other device forms, such as a head-mounted extended reality (e.g., AR or VR) device or a pair of glasses. Other devices can include mobile devices that are temporarily affixed to the body, such as for example by a user holding or strapping a smartphone or a smartwatch or a smart ring to their chest area. The motion sensor may be an accelerometer, but this disclosure contemplates that any suitable motion sensor (e.g., gyroscope, etc.) may be used, and that a combination of motion sensors may be used to detect a motion signal.

The window of data may be, e.g., one minute, although motion data may be segmented into longer or shorter windows. As described more fully below, each window of motion data may be further divided by various subprocesses.

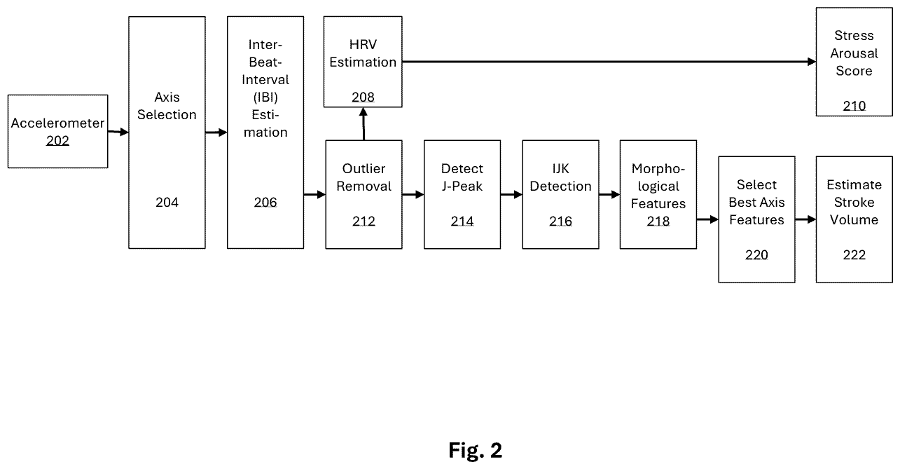

FIG. 2 illustrates an example approach for implementing the method of FIG. 1. The example of FIG. 2 includes a wearable device (e.g., an earbud) that includes an accelerometer 202.

Step 120 of the example method of FIG. 1 includes selecting motion data in the window about or more particular axes of the motion sensor. Each motion sensor has its own frame of reference, e.g., a reference set of x, y, and z axes in its own coordinate system (here, in Cartesian coordinates, although other coordinate systems may be used). Motion data is collected in this frame of reference, which may vary in how it corresponds to the frame of reference of a wearer's body. Specifically, particular embodiments evaluate the quality of acceleration data within a window by calculating the average J peak amplitude (J_{avg}) and the J peak amplitude of the ensemble-averaged BCG waveform (Jₑₙₛ) within that window. Particular embodiments may employ two thresholds to assess the quality of BCG signals for each axis. The first threshold, denoted as τₐₘₚ, is a J peak amplitude threshold and may be empirically set to 4 mG, considering that the typical BCG amplitude in the human head is approximately 10 mG. The second threshold, denoted as τ_{diff}, is applied to the difference between Jₑₙₛ and J_{avg} to discard axes with differences exceeding this threshold. For example, particular embodiments may empirically set τ_{diff} to 2 mG. Lastly, for the axes that meet the quality criteria, particular embodiments calculate the acceleration magnitude by summing their values for subsequent preprocessing and feature extraction. The following equations illustrate an example of the above description:

$$χ ( acc ) = \{ \begin{matrix}1 , if {C}_{1} and {C}_{2} \\ 0 , otherwise\end{matrix} \qquad ( 1 )$$

$${C}_{1} = {J}_{avg} \geq {τ}_{amp} \qquad ( 2 )$$

$$and$$

$${J}_{ens} \geq {τ}_{amp}$$

$${C}_{2} = \vert {J}_{avg} - {J}_{ens} \vert \leq {τ}_{diff} \qquad ( 3 )$$

$${acc}_{thresholding} = \sqrt{{\sum }_{i}^{\{ x , y , z \}} χ \cdot {acc}_{i}^{2}} \qquad ( 4 )$$

The example of FIG. 2 illustrates three example approaches for performing axis selection 204 of a motion sensor. In its simplest form, step 120 may include selecting a default axis (e.g., the x axis) of the motion sensor about which to further process motion data. However, because the motion sensor's frame of reference does not necessarily correspond to the user's body frame of reference, and further because the transformation between the relative reference frames may vary over time (e.g., because the user may rotate earbuds in the user's ear), particular embodiments may consider motion data about each of the motion sensor's axes (e.g., about each of the x, y, and z axes in the motion sensor's coordinate system) and then select one or more axes about which to further use motion data. For example, motion data about each axis that meets a quality threshold, for instance as described in the equation above, may be used for subsequent processing of the respective window of motion data.

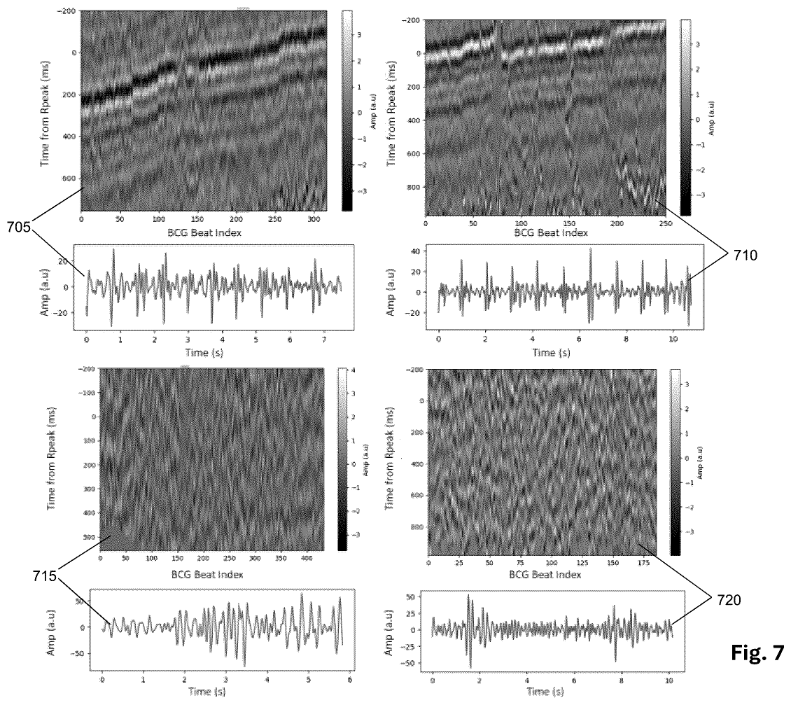

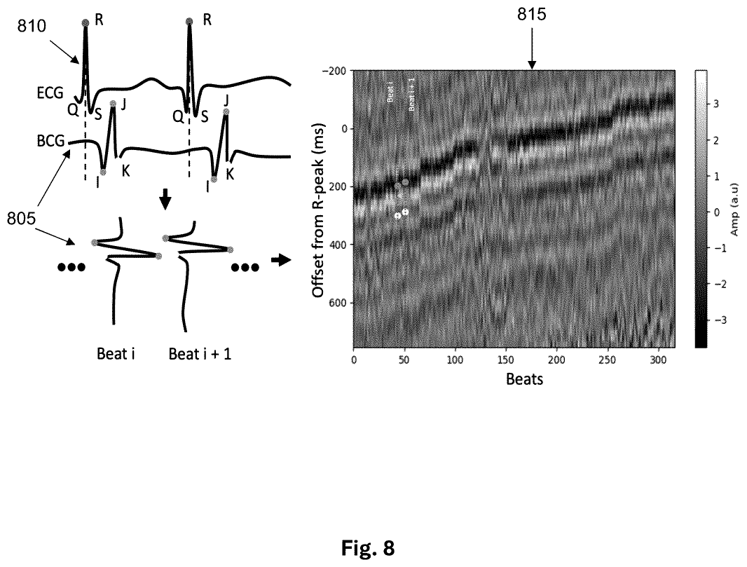

In particular embodiments, step 120 of the example method of FIG. 1 includes selecting, by a trained machine-learning model, a best axis to use for processing motion data in the respective window of motion data. For example, BCG data and ECG reference data may be simultaneously captured from a subject. One embodiment can create a heatmap by plotting the BCG beat (IJK complex) relative to the corresponding ECG beat (QRS complex) as shown in the FIG. 8. An example ECG signal 810 and an example BCG signal 805 are illustrated in FIG. 8. If BCG signal 805 is of good quality, then the BCG and ECG data will be closely correlated and a clear pattern of a certain color spectrum will emerge in heatmap 815 (e.g., the horizontal patterns in heatmap 815). After many data samples from many subjects are obtained, an expert can review the BCG signal and a corresponding ECG-BCG heat map and annotate the data, e.g., from 1 to 5. The annotations can then be fed into a machine-learning classifier along with the corresponding BCG signals as training data, in order to train the classifier to identify the quality of an input BCG signal. FIG. 7 illustrates examples of good-quality BCG signals represented by pairs of BCG-ECG heatmaps and BCG timeseries signals (pairs 705 and 710). The good quality can be explained by the heatmap patterns and clearly visible IJK complex in the BCG signal. Examples of poor-quality BCG signals represented by pairs of BCG-ECG heatmaps and BCG signals (pairs 715 and 720) which lack the clarity in heatmap patterns with less distinguishable IJK pattern in the BCG timeseries signal. Once the model is trained, then during inference BCG signals about each of the motion sensor's axes can be fed to the trained classifier, and the classifier determines which axis or axes contain good-quality motion data. In particular embodiments, motion data about more than one axis may be subsequently used, if more than axis is classified as being of sufficiently good quality, where the threshold for “sufficiently good” quality may be set automatically or by a user or medical professional.

As another example, the quality of a BCG signal for axis selection may be estimated based on a self-similarity matrix (SSM). The matrix may be input to a trained neural-network classifier, which is trained on annotated data to classify the matrix input and/or the BCG input. In addition, an SSM computation network may compute the SSM by a trained feature-extraction network, and heartbeat-related data may then be determined from the SSM by a trained heart-beat extraction network.

Step 130 of the example method of FIG. 2 includes determining a ballistocardiogram (BCG) signal in the selected motion data. BCG data represents a person's heart-beat signal based on the motion caused by the beating heart. Heart beats create motion throughout the body that can be detected by a motion sensor, but motion sensor(s) in consumer wearable device (e.g., earbuds) also detect motion from a variety of non-heart-beat related processes, such as a person talking, chewing, moving, etc. These non-heart-related motion signals can make it difficult to accurately detect BCG signals from heart beats, and therefore the BCG data must be extracted from the overall motion signal. Moreover, as discussed above, BCG signals are strongest along a particular body axis (i.e. along the foot-to-head axis), and therefore particular embodiments use axis selection to determine which wearable device axis or axes capture the strongest BCG signals. Extracting BCG signals can include step 206 of the process of FIG. 2, in which inter-beat-interval (IBI) data is estimated from the motion data about the selected axis or axes for the respective window of motion data.

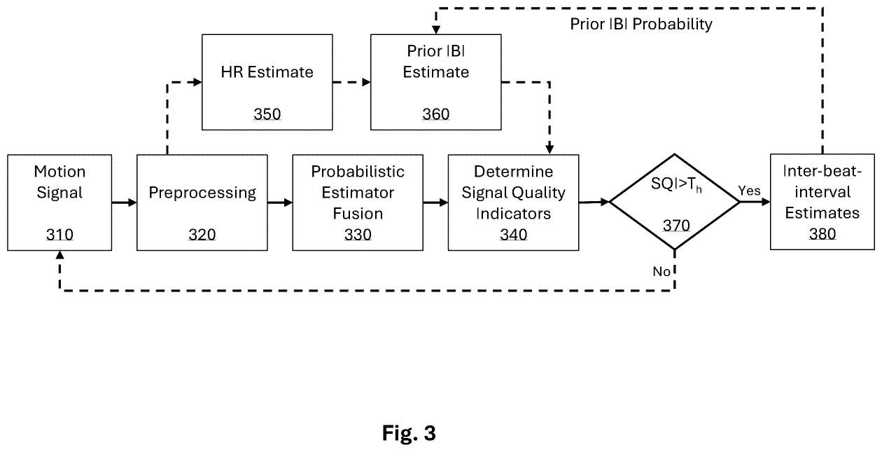

Step 140 of the example method of FIG. 1 includes determining whether the BCG signal in the selected motion data satisfies a signal-quality metric. The signal quality metric may be one or more metrics, as explained more fully herein. FIG. 3 illustrates examples of signal processing to extract BCG data from a motion signal as well as examples of signal-quality techniques to determine whether the BCG data is sufficiently accurate to make a subsequent accurate estimation of the user's stress condition.

In the example of FIG. 3, motion data 310 recorded by a motion sensor is accessed. As described above, the motion data may be segmented into windows of data, e.g., 60 seconds of data. This segmentation may occur in real time or near real time, so that the accessed 60 seconds of data is the most recently recorded data by the motion sensor, which in particular embodiments is continuously recording (e.g., at the motion sensor's sampling rate). In step 320 the motion signal undergoes processing to extract BCG data, such as the IBI signal in the window of motion data. Signal processing can include, but is not limited to, resampling and filtering of the motion signal, such as by applying a band-pass filter (e.g., from 0.5 to 15 Hz) or a Savitzky-Golay filter to the motion data in the window of motion data. In particular embodiments, as described more fully below, the window of motion data (e.g., a 60-second window of motion data) may be segmented into smaller windows of data, e.g., 3-second windows, and preprocessing 320 may be applied to each smaller window of data. These windows of data may be sliding or may be non-overlapping. As explained below, evaluating smaller windows of data within a larger window of data can improve data quality and device efficiency, for example because if a non-BCG motion dominates a motion signal for a portion of the larger window, then that portion can be discarded while retaining the other portion of high-quality data in the larger window. In particular embodiments, preprocessing 320 may be applied to a larger window of data (e.g., 60 seconds), and then this processed larger window may be segmented for further analysis, e.g., for block 330.

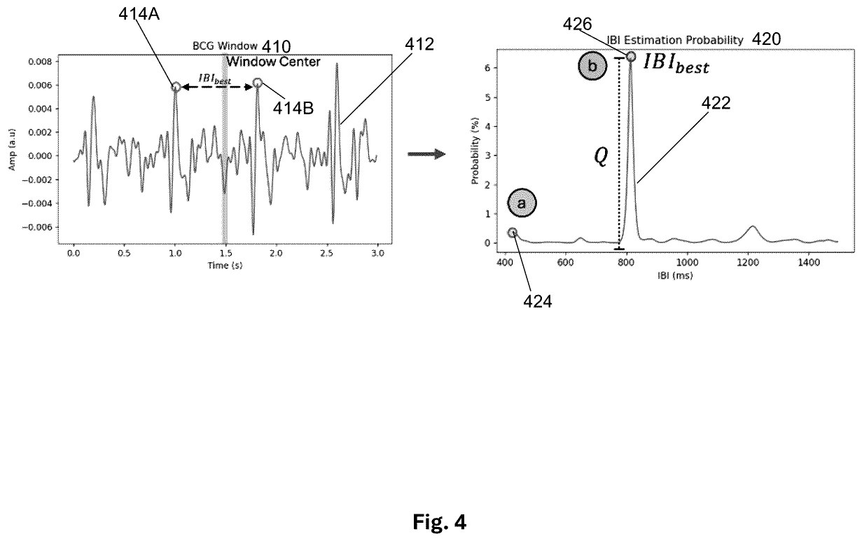

Step 330 of the example method of FIG. 3 includes applying probabilistic estimator fusion to the processed motion signal. Step 330 can include, but is not limited to, applying autocorrelation to the motion data (which may be the smaller, e.g., 3-second, window of motion data within the larger window) and/or max amplitude pair analysis. FIG. 4 illustrates an example of step 330, which may be applied to each smaller (e.g., 3-second) window of data within the larger window of motion data. FIG. 4 illustrates an example window 410 (e.g., a 3-second window) of a BCG signal 412. The BCG signal includes the IJK complex, which corresponds to stages of a heart beat. In particular, BCG signal 412 includes J-peaks such as J-peak 414A and J-peak 414B, and the distance between adjacent J-peaks (e.g., between peak 414A and peak 414B) corresponds to the inter-beat-interval between successive heartbeats. From a window of BCG data, such as window 410, step 330 may include estimating the IBI probability density function (PDF) by

$$PDF [ n ] = {S}_{Corr} [ n ] \times {S}_{AMDF} [ n ] \times {S}_{MAP} [ n ] \qquad ( 5 )$$

where S_{Corr}[n] represents a modified autocorrelation

$${S}_{Corr} [ n ] = \frac{1}{n} {\sum }_{v = 0}^{n} w \vert v \vert w [ v - N ] \qquad ( 6 )$$

S_{AMDF}[n] represents the modified average magnitude difference function

$${S}_{AMDF} [ n ] = {( \frac{1}{n} {\sum }_{v = 0}^{n} \vert w [ v ] - w [ v - n ] \vert )}^{- 1} \qquad ( 7 )$$

and S_{MAP}[n] represents the maximum amplitude pairs

$${S}_{MAP} [ n ] = ( {max}_{v ∈ \{ 0 , … n \}} ( w [ v ] + w [ v - n ] ) \qquad ( 8 )$$

The best estimate for the IBI, IBI_{best}, in the smaller window of data in the example of FIG. 4 is given by IBI_{best}=argmax (PDF), and the quality factor Q is given by Q=max(PDF). Graph 420 illustrates an example probability function 422 for window 410. As illustrated in graph 420, IBI_{best} corresponds to the maximum value of the PDF and Q is given by the amplitude of that maximum value. As illustrated in FIG. 4, an estimate 424 given by point (a) would be a probabilistically poor estimate, while an estimate 426 given by point (b) would be a relatively good estimate for the IBI from a probabilistic perspective.

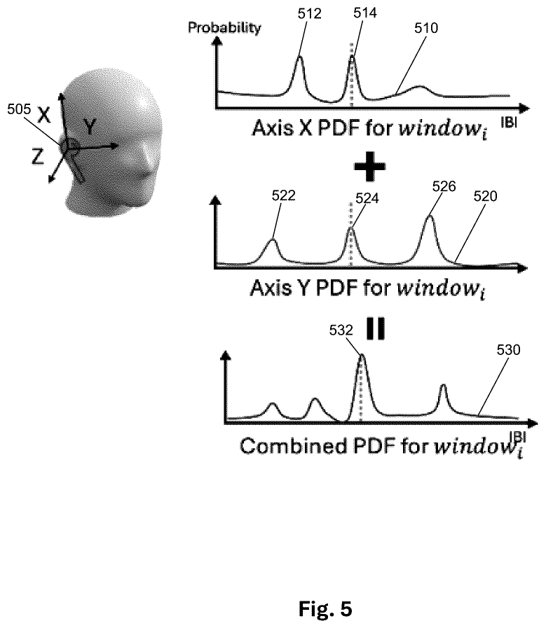

In particular embodiments step 330 may include multi-axis fusion, e.g., if multiple axes are selected in step 120 of the example method of FIG. 1. FIG. 5 illustrates an example in which data from multiple axes of earbud 505 is fused to estimate a BCG signal. Graph 510 illustrates the IBI PDF for motion corresponding to the x-axis of earbud 505. Graph 510 includes two similarly sized peaks 512 and 514, making it difficult to determine which peak corresponds to the actual IBI and which peak is spurious. Graph 520 illustrates the IBI PDF for motion corresponding to the y-axis of earbud 505, and graph 520 illustrates three peaks: peaks 522, 524, and 526, each corresponding to a different IBI probability. Graph 530 illustrates an example in which the PDF of the x-axis and the y-axis are fused, in this example by adding the two PDFs together, although other fusing approaches may be used. Graph 530 illustrates that, after fusion, peak 532 corresponds to the highest probability IBI value, and this IBI value and its corresponding Q factor in the fused signal may subsequently be used.

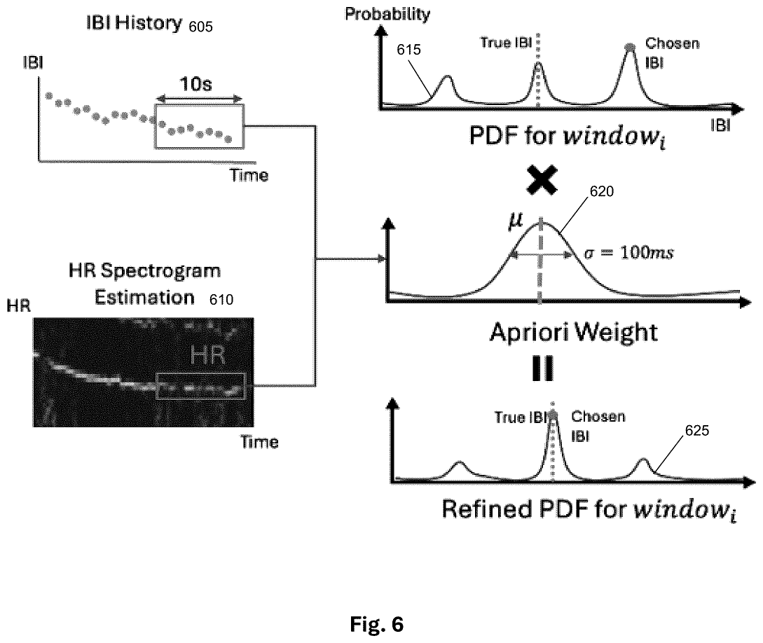

In particular embodiments, an IBI value may be estimated for a window of data using heart-rate information and a prior IBI determination. FIG. 6 illustrates an example of this IBI-estimate enhancement. As illustrated in FIG. 6, a window of data (e.g., 10 seconds) for a previous good-quality IBI determination 605 may be used as an IBI estimate prior. In addition, the user's heart rate 610 may be determined based on a window (e.g., 10 seconds) of the most recently collected motion data. The heart-rate and IBI prior may be used to determine a prior probability distribution for the user's IBI. The prior distribution is fused with the current IBI probability distribution 615 determined from the current window of data. The current IBI distribution and the prior distribution are fused, e.g., using multiplication, to determine a final PDF 625 for the current IBI estimate. As illustrated in the example of FIG. 6, a current PDF distribution 615 may include multiple probability peaks, and the highest peak may not correspond to the actual IBI value for the user. Fusing the current IBI with a prior probability distribution can improve the IBI estimated distribution, as illustrated by distribution 625, which shows that the true IBI has the highest probability after refining the current IBI estimate based on the prior distribution.

FIG. 3 illustrates an example in which step 350 performs a heart-rate (HR) estimate from a window (e.g., 10 seconds) of processed motion data. The heart rate may be estimated by any suitable approach, including a Hilbert envelope or a Fourier transform of the motion data. A prior IBI estimate is obtained in step 360, which is derived from recent (e.g., the most recent) IBI estimate that passes a quality check, such as quality evaluation 370 in the example of FIG. 3. The IBI estimate and corresponding quality metric Q can therefore be refined by weighting the current IBI estimate in light of a distribution for the IBI prior.

In the example of FIG. 3, step 340 includes determining one or more signal quality indicators that correspond to the quality of the BCG signal in the window of data. For example, step 340 may include determining whether a motion artifact is present in a smaller window that corrupts the data in that window. If so, then the data in that window may be discarded. As another example, step 340 may include a probability-density quality, such as the quality measure Q discussed above. As another example, a BCG beats per minute/heart rate (HR) ratio may be evaluated in step 340, for example by using HR data obtained in step 350. The BCG beats per minute/HR ratio should ideally be close to 1 (one), as each identified beat in the window of data should correspond to an identified IJK complex or to an identified J-peak in the BCG data.

Step 370 of the example implementation of FIG. 3 includes a evaluating a signal-quality index (SQI) to determine whether BCG data is of sufficiently high quality to use. For instance, each of the indices or metrics in step 340 may need to be satisfied in order to pass the SQI check in step 370. In particular embodiments, one or more quality metrics may need to pass a threshold Tₕ for that metric, e.g., a Q factor must be higher than a corresponding threshold value, and the max IBI in the PDF may need to be above a corresponding threshold. If a smaller window of data fails a quality check, then the data in that window may be discarded, while the data in the larger window that passes the quality check(s) is retained. In particular embodiments, if a certain amount of data (e.g., 50%) in the larger window fails a quality check, then the entire larger window of data may be discarded; otherwise, the remaining data in the larger window may be used to generate the current IBI estimate for the user.

In particular embodiments, for example as illustrated in step 212 of FIG. 2, IBI outliers in good-quality IBI data may be detected and removed. IBI outlier removal is based on the fact that consecutive beats or IBIs of a normal heart rhythm should not differ by a large margin because HR does not change rapidly over short amounts of time. The following outlier detection algorithm may be used to detect and remove IBI outliers:

Algorithm 1 IBI Outlier Detection

| Input: IBIₙ: current IBI candidate, ρₙ: peak correlation, | |
|---|---|
| IBIₙ₋₁: previous IBI candidate, IBIₙ₊₁: next IBI candidate, | |
| IBI_{avg}: running average of previous valid IBIs, and | |
| IBIₖ \| k < n: last valid IBI, Lower IBI Threshold (IBIₗ), | |
| Upper IBI Threshold (IBIᵤ), and CBD | |
| Output: IBIₙvalid: is IBIₙ valid? | |
| 1: | IBIₙvalid = false |
| 2: | if IBIₗ < IBIᵤ < IBIu then |
| 3: | if \|IBIₙ − IBIₙ₋₁ \| ≤ CBD then |
| 4: | IBIₙvalid = true |
| 5: | else if \|IBIₙ − IBIₖ \| ≤ CBD then |
| 6: | IBIₙvalid = true |
| 7: | else if \|IBIₙ − IBIₙ₊₁\| ≤ CBD & \|IBIₙ − IBI_{avg} \| ≤ |
| | CBD & ρₙ > ρ_{δ} then |
| 8: | IBIₙvalid = true |
| 9: | end if |
| 10: | end if |
| 11. | return IBIₙvalid |

In the example algorithm, the local context comparison is facilitated with the difference between current IBI and previous and next IBI values. The global context is defined by the running average of previous valid IBI values. The criterion beat different (CBD) is calculated for each input signal BCG signal from the IBI values' absolute consecutive difference (AD), and IQR is the inter-quartile range. CBD=(MED+MAD)/2, where example values include MED=3.32×IQR(AD) and MAD=(Median (IBI)×2.9×IQR(AD))/3. In the example IBI outlier detection presented above, ρₙ>ρₛ identifies a template-based correlation threshold for peak thresholds, e.g., to ensure that motion data with noise or motion artifacts is removed from the IBI data.

If a BCG signal in a larger window of data satisfies the signal-quality metric, then the motion data is used to determine one or more heart-beat metrics of the user from the selected motion data in step 150 of the example of FIG. 1. For instance, FIG. 2 illustrates an example in which heart-beat data 208 is estimated to determine a stress arousal score 210, which identifies the presence of a stress episode of the user. For example, heartbeat data may include heart-rate data and heart-rate variability data, such as Respiratory Sinus Arrythmia (RSA) metrics, although other heartbeat metrics (e.g., heart-rate variability metrics such as RMSSD, SDNN, pNN50, and LF/HF ratio) may be used. Stress arousal score 210 is based on the fact that stress exposure activates a person's sympathetic nervous system which increases heart rate and decreases heart rate variability. In particular embodiments, a stress arousal score 210 may be based on a heart-rate score and a heart-rate variability score. For example, a HR score may be calculated by (HR−70)/10.2, and an RSA score may be calculated by (RSA−7)/1.29. A reverse RSA score may then be calculated as the negative of the RSA score, and the stress arousal score 210 may be calculated as 2*(HR score)+2*(−1*RSA Score), although other score calculations including autonomic nervous system response such as systolic and diastolic blood pressure or the like may be used, and stress-arousal may be determined based on more than two HR/HRV inputs. Stress arousal score 210 is one example of step 160 of the example method of FIG. 1, which includes estimating, based on the determined heart-beat metrics, a stress condition of the user at a time coincident with the window of motion data. In other words, the stress condition may be a stress arousal of the user.

In particular embodiments, an estimated stress condition in step 160 of the example of FIG. 1 may be, or may include, estimating advanced biomarkers than are indicative of positive or negative stress, e.g., can be used to classify a stress arousal incident as “healthy” stress or as “unhealthy” stress. For instance, in the example of FIG. 2, the J-peaks in a window of data may be detected in step 214. The corresponding IJK complexes in the window of data may then be detected in step 216, for example by using ensemble techniques or averaging techniques. In step 218, morphological features are then extracted from the data window. In each window of data, each particular feature in that window may be identified and then averaged to get the value for that feature for that window. Examples of morphological features extracted from the BCG data include J-peak amplitude, negative amplitude (I-peak amplitude), peak-to-peak amplitude (I-to-J peak amplitude), maximum amplitude, IBI, RMS, and other statistical measure such as standard deviations, etc. of some or all of these features. In particular embodiments, in step 220 the features about the best axis or axes may be used, as described above in connection with axis-selection embodiments. These features are selected based on univariate correlation with stroke volume, which has been shown to be related to negative and positive stress classification.

Identified features are used to train a regression model to estimate stroke volume, in particular embodiments and as in step 222 of the implementation of FIG. 2, or to estimate cardiac output directly, in other embodiments. In the former embodiments, cardiac output CO may be determined by CO=HR×(stroke volume). The regression model is trained using, e.g., supervised training with input features and corresponding labelled stress events, where the labels may be determined based on self-reporting and/or performance of pre-defined tasks. Cardiac output may also be correlated to stress-event classification in that cardiac output tends to increase relative to baseline during a positive stress event while cardiac output does not increase (or increase as much) relative to baseline for a negative stress event. Therefore, stress events may be classified based on the determined cardiac output relative to baseline, and the cardiac output can be determined based on morphological features extracted from BCG signals obtained from motion data of a motion sensor of a consumer wearable device.

The above examples describe estimating heart-beats metrics such as IBI from BCG data obtain by a motion sensor. Other embodiments may in addition, or alternatively, determine heart-beat metrics such as IBI from photoplethysmography (PPG) data collected by a PPG sensor, which uses an optical signal to detect blood-volume changes in tissue. The PPG sensor may be part of the same wearable device that contains the motion sensor or may be part of a different wearable device.

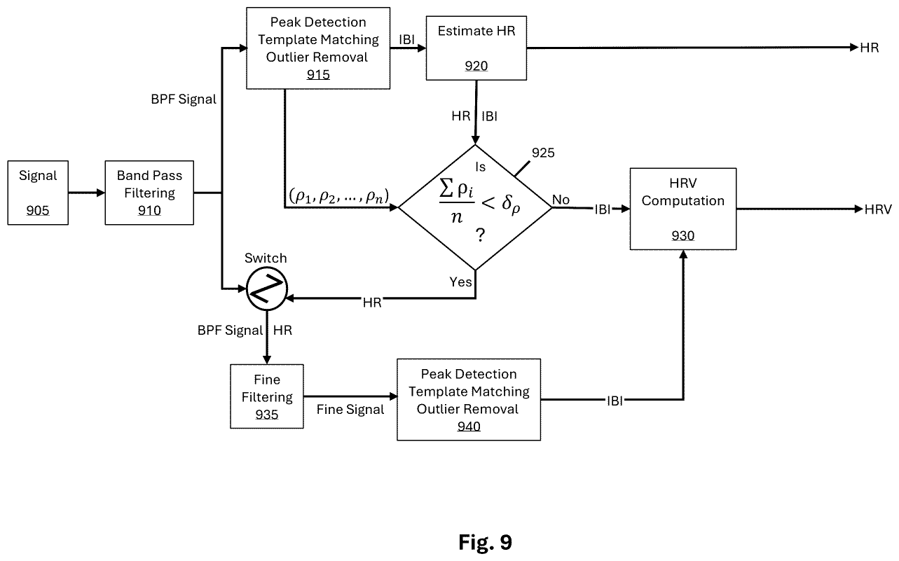

FIG. 9 illustrates an example process for estimating IBI and corresponding heart-beat metrics, such as HR and HRV, based on a PPG signal. In the example of FIG. 9, a PPG signal obtained from a PPG sensor is filtered in step 910, for example using third-order Butterworth filter with a cut-off frequency range [0.7 Hz, 3.5 Hz]. The filtered signal may be divided into segments (e.g., 1-minute segments) with or without overlap. The filtered signal is then sent to block 915, which performs peak detection in the PPG data (which may be a window of PPG data), template matching, and outlier removal to estimate the IBI of the user. From the IBI signal, step 920 estimates the heart rate (HR) of the user. Peak detection may be determined by, for example a sign reversal check between the difference in sample values before and after the peak.

Quality estimation of a PPG signal may be performed using template matching, which quantifies regularity in a signal by comparing the signal with a template signal. To estimate a template peak, IBI values are first calculated from detected peak position of the input PPG signal. The median IBI value is multiplied by the sampling frequency to obtain the number of samples (k) in one PPG peak pulse. The peak pulse of size (k) is extracted from each detected PPG peak. The average of all peak pulses is the template peak segment. Then, the Pearson correlation between each PPG peak pulse and the template segment is computed to obtain the average template matching correlation.

To determine whether an IBI estimate from PPG data is of sufficient quality to estimate heart-rate variability, step 925 of the example of FIG. 9 determines whether the average correlation coefficient

$$\frac{\sum {ρ}_{i}}{n}$$

is below a threshold δₚ, where ρᵢ is the correlation coefficient for the ith peak. In particular embodiments, a threshold value of 0.9 may be used, which experimentally may minimize errors of various HRV features. If the average correlation coefficient is not less than the threshold, then the PPG signal is of sufficient quality to determine HRV directly, and step 930 includes determining the HRV from the PPG signal. Otherwise, if the average correlation coefficient is less than the threshold, then fine filtering 935 is needed to refine the PPG data and accurately detect HRV for the user. To perform fine filtering, the PPG signal is band-passed filtered using the estimated HR as the center frequency and a range (e.g., plus or minus 0.2 Hz) around that center frequency. The fine filtered signal is then used to perform peak detection, template matching, and outlier removal in step 940, and the resulting fine-filtered IBI signal is used to perform HRV computation 930. In the example of FIG. 9, outlier removal may be performed using, e.g., the outlier removal algorithm described above.

The example implementation of FIG. 9 uses a template-matching approach for checking the quality of a PPG signal. Other embodiments additionally or alternatively use morphological features to classify the PPG signal quality. For example, a good-quality signal may show a systolic peak, the diastolic peak, and a dicrotic notch for each PPG peak. An acceptable signal may show only a systolic peak, which is sufficient for determining IBI from PPG signals, while a poor (unacceptable) signal may not readily evidence a systolic peak.

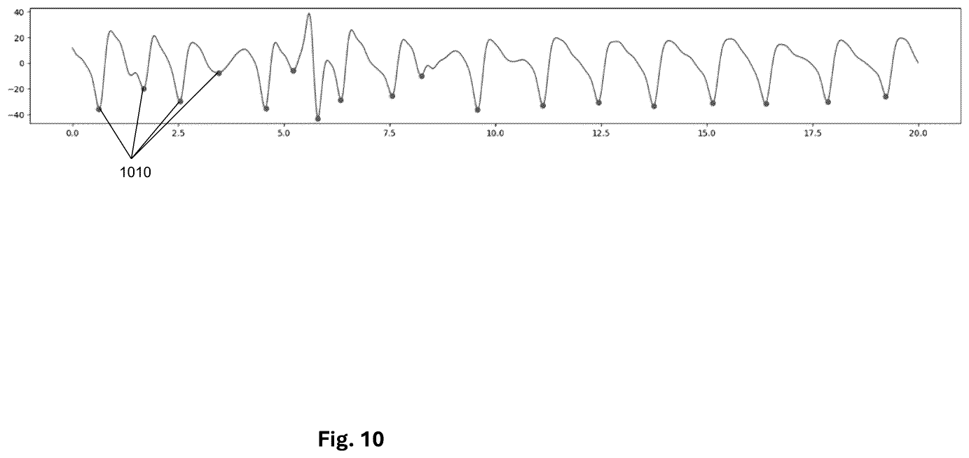

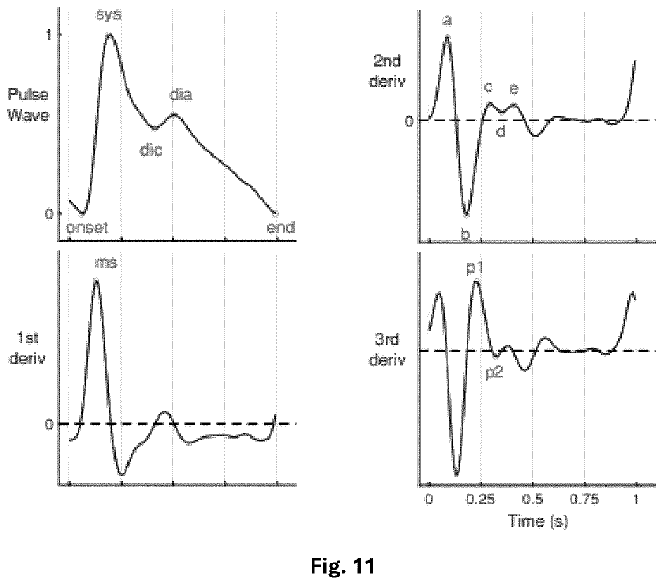

To extract morphological features, particular embodiments identify each “foot” in a PPG signal, and the foot-to-foot signal is one PPG cycle. FIG. 10 illustrates an example PPG signal illustrating multiple feet 1010, which are some of the feet in the overall signal shown in FIG. 10. Then, features from each cycle can be extracted to identify the quality of the PPG signal. For example, for each peak, features can be extracted from the PPG signal and from each of the 1^{st}, 2^{nd}, and 3^{rd} derivatives of that signal. FIG. 11 illustrates an example of a high-quality PPG signal and corresponding derivatives. Fiducial points in the PPG signal are inflection points corresponding to zero-crossing points in the derivatives. Morphological features are identified in FIG. 11 as follows. In the original PPG signal pulse wave, the PPG cycle illustrates a defined onset, a systolic peak, a dicrotic notch, a diastolic peak, and an end. The systolic peak is the maximum of the pulse-wave signal. The first derivative includes a maximum ms, where the time at which ms occurs is before the time at which the systolic peak occurs in the pulse wave, i.e., t(ms)<t(sys). The 2_{nd} derivative includes 5 fiducial points a, b, c, d, and e. The third derivative includes points p1 and p2. An excellent PPG signal would include all of these features in its pulse-wave signal and the corresponding derivatives. However, an acceptable signal may include only some features, such as an onset, a systolic peak, and an end in the pulse-wave signal; the presence of a maximum ms in the first derivative that precedes the systolic peak; points a, b, and c in the 2nd derivative; and either p1 or p2 in the third derivative. In particular embodiments, PPG data may be classified as, e.g., excellent, acceptable, and unusable based on these morphological features, and the classified data and features may be used to train a classifier to output the quality of an input PPG signal. Thus, once PPG cycles are identified in a PPG signal, then the morphological features for each cycle may be determined and input to a trained classifier trained to classify the quality of a PPG signal.

In particular embodiments, a user's device(s) may be passively sensing IBI using both PPG and BCG pathways, whether the motion sensor and PPG sensor are in the same wearable device or in different wearable devices. In these embodiments, the IBI estimates from the different sensor modalities may be fused to improve IBI detection and subsequent heart-beat-related information, such as HR and HRV, to predict stress arousal. This fusion can be performed minute by minute or other time window based on the use case or applications.

As an example of sensor fusion, if one of the BCG signal and the PPG signal exceeds a quality threshold, then that signal may be used to determine the user's current IBI and heat-beat related information. As another example, if both PPG and BCG biomarkers are above the quality threshold for a particular time window, then the modality with the best quality estimate can be selected. As another example, if both PPG and BCG biomarkers are above the quality threshold for a particular time window, then particular embodiments may take the aggregated (e.g., mean) value of each biomarker and, in particular embodiments, then apply Kalman Filter to reduce any sudden large variations.

If a stress episode is detected, and in particular a negative stress episode, then subsequent functionality can include notifying the user and/or a medical professional, and/or providing stress management solutions to the user (e.g., in the form of guided breathing, nerve stimulation, environmental changes such as soothing music, etc.). For example, a notification may be surfaced to a user's smartphone or wearable device to immediately perform breathing techniques to reduce the user's stress.

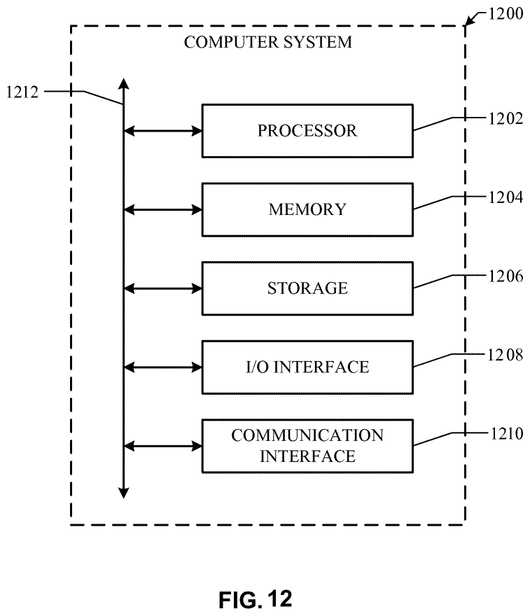

FIG. 12 illustrates an example computer system 1200. In particular embodiments, one or more computer systems 1200 perform one or more steps of one or more methods described or illustrated herein. In particular embodiments, one or more computer systems 1200 provide functionality described or illustrated herein. In particular embodiments, software running on one or more computer systems 1200 performs one or more steps of one or more methods described or illustrated herein or provides functionality described or illustrated herein. Particular embodiments include one or more portions of one or more computer systems 1200. Herein, reference to a computer system may encompass a computing device, and vice versa, where appropriate. Moreover, reference to a computer system may encompass one or more computer systems, where appropriate.

This disclosure contemplates any suitable number of computer systems 1200. This disclosure contemplates computer system 1200 taking any suitable physical form. As example and not by way of limitation, computer system 1200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1200 may include one or more computer systems 1200; be unitary or distributed; span multiple locations; span multiple machines; span multiple data centers; or reside in a cloud, which may include one or more cloud components in one or more networks. Where appropriate, one or more computer systems 1200 may perform without substantial spatial or temporal limitation one or more steps of one or more methods described or illustrated herein. As an example and not by way of limitation, one or more computer systems 1200 may perform in real time or in batch mode one or more steps of one or more methods described or illustrated herein. One or more computer systems 1200 may perform at different times or at different locations one or more steps of one or more methods described or illustrated herein, where appropriate.

In particular embodiments, computer system 1200 includes a processor 1202, memory 1204, storage 1206, an input/output (I/O) interface 1208, a communication interface 1210, and a bus 1212. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1202 includes hardware for executing instructions, such as those making up a computer program. As an example and not by way of limitation, to execute instructions, processor 1202 may retrieve (or fetch) the instructions from an internal register, an internal cache, memory 1204, or storage 1206; decode and execute them; and then write one or more results to an internal register, an internal cache, memory 1204, or storage 1206. In particular embodiments, processor 1202 may include one or more internal caches for data, instructions, or addresses. This disclosure contemplates processor 1202 including any suitable number of any suitable internal caches, where appropriate. As an example and not by way of limitation, processor 1202 may include one or more instruction caches, one or more data caches, and one or more translation lookaside buffers (TLBs). Instructions in the instruction caches may be copies of instructions in memory 1204 or storage 1206, and the instruction caches may speed up retrieval of those instructions by processor 1202. Data in the data caches may be copies of data in memory 1204 or storage 1206 for instructions executing at processor 1202 to operate on; the results of previous instructions executed at processor 1202 for access by subsequent instructions executing at processor 1202 or for writing to memory 1204 or storage 1206; or other suitable data. The data caches may speed up read or write operations by processor 1202. The TLBs may speed up virtual-address translation for processor 1202. In particular embodiments, processor 1202 may include one or more internal registers for data, instructions, or addresses. This disclosure contemplates processor 1202 including any suitable number of any suitable internal registers, where appropriate. Where appropriate, processor 1202 may include one or more arithmetic logic units (ALUs); be a multi-core processor; or include one or more processors 1202. Although this disclosure describes and illustrates a particular processor, this disclosure contemplates any suitable processor.

In particular embodiments, memory 1204 includes main memory for storing instructions for processor 1202 to execute or data for processor 1202 to operate on. As an example and not by way of limitation, computer system 1200 may load instructions from storage 1206 or another source (such as, for example, another computer system 1200) to memory 1204. Processor 1202 may then load the instructions from memory 1204 to an internal register or internal cache. To execute the instructions, processor 1202 may retrieve the instructions from the internal register or internal cache and decode them. During or after execution of the instructions, processor 1202 may write one or more results (which may be intermediate or final results) to the internal register or internal cache. Processor 1202 may then write one or more of those results to memory 1204. In particular embodiments, processor 1202 executes only instructions in one or more internal registers or internal caches or in memory 1204 (as opposed to storage 1206 or elsewhere) and operates only on data in one or more internal registers or internal caches or in memory 1204 (as opposed to storage 1206 or elsewhere). One or more memory buses (which may each include an address bus and a data bus) may couple processor 1202 to memory 1204. Bus 1212 may include one or more memory buses, as described below. In particular embodiments, one or more memory management units (MMUs) reside between processor 1202 and memory 1204 and facilitate accesses to memory 1204 requested by processor 1202. In particular embodiments, memory 1204 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1204 may include one or more memories 1204, where appropriate. Although this disclosure describes and illustrates particular memory, this disclosure contemplates any suitable memory.

In particular embodiments, storage 1206 includes mass storage for data or instructions. As an example and not by way of limitation, storage 1206 may include a hard disk drive (HDD), a floppy disk drive, flash memory, an optical disc, a magneto-optical disc, magnetic tape, or a Universal Serial Bus (USB) drive or a combination of two or more of these. Storage 1206 may include removable or non-removable (or fixed) media, where appropriate. Storage 1206 may be internal or external to computer system 1200, where appropriate. In particular embodiments, storage 1206 is non-volatile, solid-state memory. In particular embodiments, storage 1206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1206 taking any suitable physical form. Storage 1206 may include one or more storage control units facilitating communication between processor 1202 and storage 1206, where appropriate. Where appropriate, storage 1206 may include one or more storages 1206. Although this disclosure describes and illustrates particular storage, this disclosure contemplates any suitable storage.

In particular embodiments, I/O interface 1208 includes hardware, software, or both, providing one or more interfaces for communication between computer system 1200 and one or more I/O devices. Computer system 1200 may include one or more of these I/O devices, where appropriate. One or more of these I/O devices may enable communication between a person and computer system 1200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1208 for them. Where appropriate, I/O interface 1208 may include one or more device or software drivers enabling processor 1202 to drive one or more of these I/O devices. I/O interface 1208 may include one or more I/O interfaces 1208, where appropriate. Although this disclosure describes and illustrates a particular I/O interface, this disclosure contemplates any suitable I/O interface.

In particular embodiments, communication interface 1210 includes hardware, software, or both providing one or more interfaces for communication (such as, for example, packet-based communication) between computer system 1200 and one or more other computer systems 1200 or one or more networks. As an example and not by way of limitation, communication interface 1210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1210 for it. As an example and not by way of limitation, computer system 1200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1200 may include any suitable communication interface 1210 for any of these networks, where appropriate. Communication interface 1210 may include one or more communication interfaces 1210, where appropriate. Although this disclosure describes and illustrates a particular communication interface, this disclosure contemplates any suitable communication interface.

In particular embodiments, bus 1212 includes hardware, software, or both coupling components of computer system 1200 to each other. As an example and not by way of limitation, bus 1212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1212 may include one or more buses 1212, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
accessing a window of motion data collected by a motion sensor of a device worn by a user;
selecting motion data in the window about or more particular axes of the motion sensor;
determining a ballistocardiogram (BCG) signal in the selected motion data;
determining whether the BCG signal in the selected motion data satisfies a signal-quality metric; and
in response to a determination that the BCG signal in the selected motion data satisfies the signal-quality metric, then:
determining one or more heart-beat metrics of the user from the selected motion data; and
estimating, based on the one or more determined heart-beat metrics, a stress condition of the user at a time coincident with the window of motion data.

2. The method of claim 1, wherein the one or more heart-beat metrics of the user comprise a heart-rate metric and a heart-rate-variability metric of the user; and
the stress condition comprises a stress arousal level of the user.

3. The method of claim 1, wherein determining one or more heart-beat metrics of the user from the selected motion data comprises estimating, in the selected motion data, an inter-beat-interval (IBI) of the user at the time coincident with the window of motion data.

4. The method of claim 3, wherein estimating the IBI of the user comprises:
determining a probability distribution of an IBI value from the BCG signal;
determining a prior IBI probability distribution based on (1) a heart rate of the user and (2) a recent prior IBI estimate for the user; and
estimating the IBI of the user by weighting the probability distribution of the IBI value from the BCG signal with the prior IBI probability distribution.

5. The method of claim 3, wherein determining whether the BCG signal in the selected motion data satisfies a signal-quality metric comprises determining whether the BCG signal in the selected motion data satisfies a criteria for each of:
a probability distribution of the IBI;
an amount of the BCG signal containing one or more motion artifacts; and
a ratio of a number of heart beats detected in the IBI to the number of heart beats detected by an HR estimated from the BCG signal.

6. The method of claim 3, wherein selecting motion data in the window about or more particular axes of the motion sensor comprises determining that motion data about each of a plurality of axes exceeds a quality threshold; and
estimating the IBI of the user comprises:
determining, for each of the plurality of axes, an IBI probability distribution from the BCG signal corresponding to that axis;
combining each IBI probability distribution into a single combined IBI probability distribution; and
selecting, as the IBI estimate, the IBI value corresponding to the highest probability in the combined IBI probability distribution.

7. The method of claim 3, wherein selecting motion data in the window about or more particular axes of the motion sensor comprises:
providing a BCG signal corresponding to each particular axis of the motion sensor to a classifier trained on annotated BCG data; and
determining, by the classifier, whether the BCG signal about each particular axis of the motion sensor comprises a high-quality BCG signal.

8. The method of claim 3, wherein selecting motion data in the window about or more particular axes of the motion sensor comprises:
determining, for each BCG signal determined from motion data about each axis of the motion sensor, a self-similarity matrix; and
determining, based on each self-similarity matrix, whether the BCG signal about each axis of the motion sensor comprises a high-quality BCG signal.

9. The method of claim 3, further comprising:
determining, from the BCG signal, a plurality of features corresponding to a plurality of IJK complexes in the BCG signal;
determining, by a trained machine-learning model and based on the plurality of features, whether the stress condition of the user corresponds to a negative stress event or a positive stress event.

10. The method of claim 9, wherein the trained machine learning model outputs a stroke volume of the user.

11. The method of claim 9, wherein the trained machine learning model outputs a cardiac output of the user.

12. The method of claim 1, wherein the device worn by the user comprises a head-worn device.

13. The method of claim 12, wherein the head-worn device comprises one or more earbuds.

14. The method of claim 1, further comprising:
accessing photoplethysmography (PPG) data collected by a PPG sensor of a PPG device worn by the user;
determining the one or more heart-beat metrics of the user from peaks in the accessed PPG data; and
determining whether to estimate the stress condition of the user based on the BCG signal, the PPG data, or both.

15. The method of claim 14, wherein determining whether to estimate the stress condition of the user based on the BCG signal, the PPG data, or both comprises:
determining a first quality score for the BCG signal and a second quality score for the PPG data; and
selecting the BCG signal or the PPG data based on whether the first quality score is higher than the second quality score.

16. The method of claim 14, wherein determining whether to estimate the stress condition of the user based on the BCG signal, the PPG data, or both comprises:
determining a first quality score for the BCG signal and a second quality score for the PPG data; and
when both the first quality score and the second quality score exceed a respective score threshold, then aggregating the one or more heart-beat metrics from the PPG data and the BCG signal and estimating the stress condition of the user based on the aggregated one or more metrics.

17. An apparatus comprising: one or more non-transitory computer readable storage media storing instructions; and one or more processors coupled to the one or more non-transitory computer readable storage media and operable to execute the instructions to:
access a window of motion data collected by a motion sensor of a device worn by a user;
select motion data in the window about or more particular axes of the motion sensor;
determine a ballistocardiogram (BCG) signal in the selected motion data;
determine whether the BCG signal in the selected motion data satisfies a signal-quality metric; and
in response to a determination that the BCG signal in the selected motion data satisfies the signal-quality metric, then:
determine one or more heart-beat metrics of the user from the selected motion data; and
estimate, based on the one or more determined heart-beat metrics, a stress condition of the user at a time coincident with the window of motion data.

18. The apparatus of claim 17, wherein determining one or more heart-beat metrics of the user from the selected motion data comprises estimating, in the selected motion data, an inter-beat-interval (IBI) of the user at the time coincident with the window of motion data.

19. One or more non-transitory computer readable storage media storing instructions that are operable when executed to:
access a window of motion data collected by a motion sensor of a device worn by a user;
select motion data in the window about or more particular axes of the motion sensor;
determine a ballistocardiogram (BCG) signal in the selected motion data;
determine whether the BCG signal in the selected motion data satisfies a signal-quality metric; and
in response to a determination that the BCG signal in the selected motion data satisfies the signal-quality metric, then:
determine one or more heart-beat metrics of the user from the selected motion data; and
estimate, based on the one or more determined heart-beat metrics, a stress condition of the user at a time coincident with the window of motion data.

20. The media of claim 19, wherein determining one or more heart-beat metrics of the user from the selected motion data comprises estimating, in the selected motion data, an inter-beat-interval (IBI) of the user at the time coincident with the window of motion data.
